(19) United States
(12) Patent Application Publication
(10) Pub. No.: US 2022/0031517 A1
(43) Pub. Date: Feb. 3, 2022

(54) ELECTRONIC HEARING PROTECTION HAVING IMPROVED NOISE REDUCTION

(71) Applicant: AOB Products Company, Columbia, MO (US)

(72) Inventors: Kyle Martin, Columbia, MO (US); James Tayon, Moberly, MO (US); Michael Cottrell, Ashland, MO (US)

(21) Appl. No.: 17/387,998

(22) Filed: Jul. 28, 2021

Related U.S. Application Data
(60) Provisional application No. 63/057,604, filed on Jul. 28, 2020.

(51) Int. Cl. A61F 11/08; H04R 1/10; H04R 31/00

(52) U.S. Cl. CPC A61F 11/08; A61F 2011/085; H04R 31/00; H04R 1/1016

(57) ABSTRACT

Electronic hearing protection earwear having increased noise reduction rating. The earwear can be configured to plug an ear of the user. The earwear includes a housing defining an interior in which electronic components of the earwear are received. Void filling material can be installed in the housing interior to reduce vibration and sound wave propagation, thus increasing the noise reduction rating of the earwear.

### CROSS-REFERENCE TO RELATED APPLICATION

The present application claims priority to U.S. Provisional Patent App. No. 63/057,604, filed Jul. 28, 2020, which is hereby incorporated by reference in its entirety for all purposes.

### FIELD

The present disclosure generally relates to earwear for humans, and more particularly to electronic earwear, such as electronic hearing protection.

### BACKGROUND

People use various types of earwear for hearing protection and other purposes. Some types of earwear include ear buds, ear muffs, and head phones.

### SUMMARY

In one aspect, an electronic ear plug is for protecting an ear drum of a human from ambient sound waves. The ear plug comprises a housing defining an interior. The ear plug includes a tip supported by the housing. The tip is configured to be received in an ear canal of the human. A sound wave passage extends through the ear tip. A speaker in the housing is configured to emit sound waves to be delivered through the sound wave passage toward the ear drum. A sealing material in the housing at least partially fills the housing. The sealing material is configured to obstruct sound waves not emitted by the speaker from entering the sound wave passage from inside the housing.

In another aspect, a hearing protection device is for protecting an ear drum of a human. The device comprises a housing configured to be worn by the human. The device further comprises a speaker in the housing. The speaker is configured to emit sound waves toward the ear drum of the human when the housing is worn by the human. A sound wave passage is configured to permit passage of sound waves emitted by the speaker to the ear drum of the user when the housing is worn by the human. A sealing material in the housing at least partially fills the housing. The sealing material is configured to obstruct sound waves not emitted by the speaker from entering the sound wave passage from inside the housing.

In yet another aspect, a method of manufacturing an ear plug comprises arranging a speaker in a housing of the ear plug to emit sound waves through a sound wave passage of the ear plug. The method further comprises installing sealing material in an interior of the housing. The sealing material forms a peripheral seal around the speaker and at least partially fills a void to a side of the speaker opposite the sound wave passage. The method further comprises permitting the sealing material to stiffen.

Other objects and features of the present disclosure will be in part apparent and in part pointed out herein.

### BRIEF DESCRIPTION OF THE DRAWINGS

FIG. 1 is a perspective of an electronic ear plug of the present disclosure;

FIG. 2 is a view similar to FIG. 1 but of the ear plug having an ear tip thereof removed;

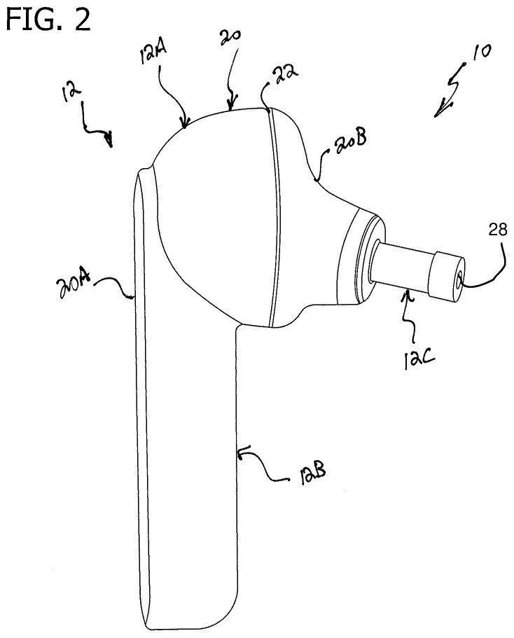

FIG. 3 is a perspective of the electronic ear plug having a portion of a housing thereof broken away to show an interior of the housing;

FIG. 4 is a view similar to FIG. 3 but showing fill material in the interior of the housing;

FIG. 5 is a perspective of the electronic ear plug showing fill material in two components of the housing before the housing is assembled to with the fill material therein; and

FIG. 6 is a schematic of a control system of the electronic ear plug.

Corresponding reference numbers indicate corresponding parts throughout the drawings.

### DETAILED DESCRIPTION

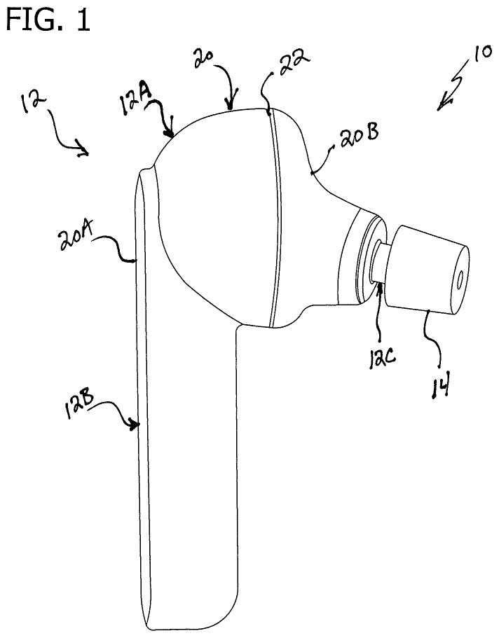

Referring to FIG. 1, an electronic ear bud (broadly, “earwear” or “in-ear earwear”) of the present disclosure is indicated by the reference number 10. Although one ear bud is shown, it will be understood that usually a set of two ear buds would be provided, one for each ear of a user. The ear buds of a set could be mirror images of each other or have identical or different construction.

As explained in further detail below, the ear bud 10 is configured as an ear plug and can be used to protect the ear drum of a user from undesired or harmful sound waves and/or to deliver desired sound waves to the ear drum of the user. In one aspect, the ear bud 10 can be referred to broadly as hearing protection. However, it will be understood that earwear according to this disclosure may not be used for hearing protection. Moreover, the earwear may have a different configuration, and may not be configured for delivering sound waves to the ear drum.

Referring to FIG. 1, the ear bud 10 includes a body 12 and an ear tip 14 connected to the body. The body 12 includes a bulbous head 12A and an arm 12B extending downward from the head. The body 12 includes a laterally protruding stem 12C on which the ear tip 14 is received to connect the ear tip to the body. The stem 12C releasably mates with an opening in the ear tip 14 to mount the ear tip on the stem. In use, the ear tip 14, on the stem 12C, is installed in an ear canal of a user, and the head 12A of the body 12 can be received in the outer ear of the user. In this manner, the ear bud 10 is supported on the ear of the user.

The ear tip 14 is desirably sized and shaped to fit into the ear canal to plug the ear canal by forming a circumferential seal with the ear canal. The ear tip 14, forming a seal with the ear canal, blocks air and sound waves from moving along the ear canal past the ear tip to the ear drum. For example, the ear tip 14 can be formed of an elastomeric material (e.g., silicone) that is resiliently compressible and form-fitting to the ear canal. A kit of ear tips having different sizes and/or shapes may be provided so the user can select an ear tip having a desired fit in their ear canal and/or in the outer ear.

The ear bud 10 includes a housing 20 forming the body of the ear bud. The housing includes a first housing portion 20A and a second housing portion 20B. The first housing portion 20A forms the arm 12B and forms a first segment of the head 12A. The second housing portion 20B forms a second segment of the head 12A and forms the stem 12C. The first and second housing portions 20A, 20B are configured to be connected to each other to assemble the body 12. More specifically, the second housing portion 20B has a circular flange receivable in the opening of the first housing portion 20A to define a joint 22 connecting the housing portions. The housing 20 defines an interior in which electronic components of the ear bud are received, as explained in further detail below.

The housing interior includes a first interior region 26A in the head 12A and a second interior region 26B in the arm 12B. A sound wave passage 28 in the stem 12C extends from the first interior region 26A to an opening in a free end of the stem. The electronic components of the ear bud include a battery 30 (broadly, “power source”) and a speaker 32 (broadly, “sound wave emitter”) connected to the battery for powering the speaker. The battery 30 and speaker 32 are received in the first interior region 26A. In particular, the speaker 32 is received in the first interior region 26A near a proximal end of the stem 12C at a connection of the head 12A to the stem. The speaker 32 is oriented to emit sound waves into the passage of the stem 12C to exit the open free end of the stem into the ear canal of the wearer. For example, the speaker 32 may produce sound waves to cause the user to hear music, reproduced ambient or environmental sounds, audio from a telephone connected to the ear bud, etc.

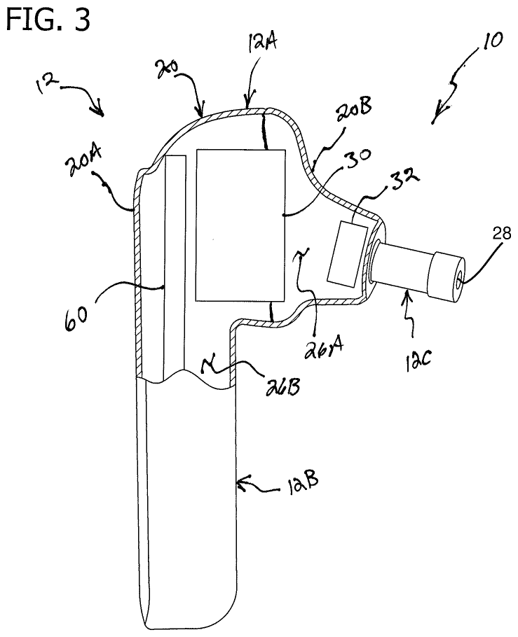

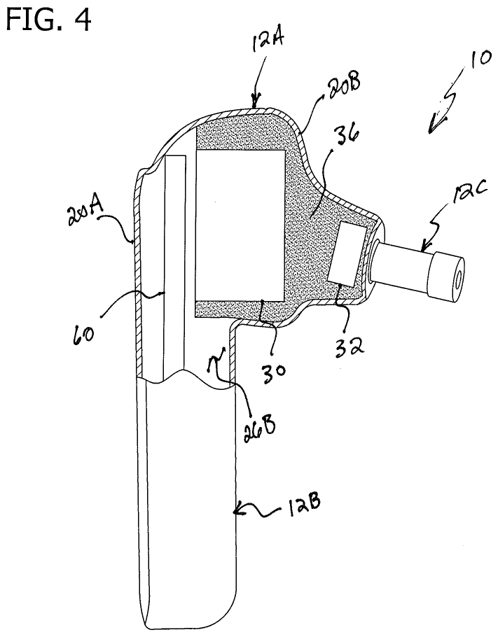

As shown in FIG. 4, the ear bud 10 includes a sealing and acoustic dampening fill material 36 in the interior of the body 12. As shown by comparison of FIGS. 3 and 4, the fill material 36 fills a substantial volume of the interior of the head 12A. The fill material 36 creates an air tight seal around the speaker 32 and stem 12C to prevent entry of ambient sound and sound propagation by air into the ear canal. The fill material 36 also provides general acoustic dampening within the body of the electronic ear bud 10 to reduce acoustic vibration and sound propagation into the ear canal through the stem 12C. For example, the sealing and acoustic dampening material 36 can comprise silicone and/or another suitable material provided in (filling at least a portion of) the interior of the ear bud 10. Desirably, the sealing material fills substantially all (at least 80%) of the first interior region 26A.

The combined effect of air sealing and acoustic dampening provided by the fill material 36 aids in improving the noise reduction rating (NRR) of the ear bud 10. Without the void filling material 36 in the interior of the housing, sound waves might pass into the housing and through the stem 12C into the ear canal. The void filling material 36 blocks such sound waves and dampens vibration of the ear bud 10. This results in increased NRR for the ear bud. The fill material 36 reduces the capability of the stem 12C to transmit vibration or ambient sound waves through the opening in the ear tip 14 used to mount the ear tip on the stem. In testing, the fill material 36 increased the NRR for the ear buds to 23 decibels. Desirably, the NRR is increased to at least 18 decibels, more desirably at least 20 decibels, and even more desirably at least 22 decibels.

Various void filling materials 36, such as silicon-based caulk, latex-based caulk, or foam (e.g., urethane foam) could be used. Desirably, the void filling material 36 (or substance) can be installed (e.g., inserted, injected, poured, etc.) into the interior of the housing in an uncured or unhardened state (broadly, “first state”) so the material more easily conforms to and fills voids. The material 36 is then permitted to cure or stiffen (broadly, “second state”) as installed to remain in place and provide the desired noise blockage and vibration attenuation. The void filling material 36 substantially fills gaps between interior components (e.g., electronics) and interior surfaces of the housing 20. The void filling material 36 can be provided in the housing at least in a portion of the housing that overlies the entrance of the user's ear canal in use. Desirably, but not necessarily, the void filling material 36 in its installed state (second state) is resiliently compressible and not rigid to facilitate vibration and acoustic dampening.

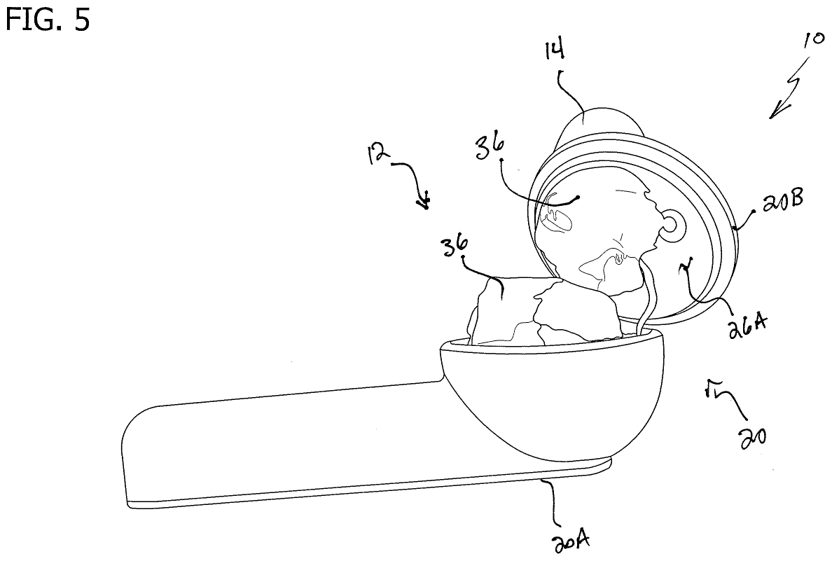

In one example, the sealing and acoustic dampening material 36 can be installed on interiors of one or more housing parts 20A, 20B before the housing 20 is closed. For example, referring to FIG. 5, void filling material 36 in the form of caulk or paste can be applied directly to the speaker 32 and in the openings of the housing portions 20A, 20B. The paste is spread liberally and packed around the speaker 32 to close and seal air gaps around the speaker. The paste covers a back side of the speaker that faces away from the sound wave passage 28. The paste also forms a peripheral seal around the speaker, and fills gaps between the speaker and interior surfaces of the housing 20. While the material is still uncured, the housing assembly 20 is put together, allowing excess filling material 36 to flow around internal components, primarily the battery 30, displacing most of the air that would be otherwise enclosed in the housing. Optionally, caulk or paste 36 can be applied to the interiors of both housing parts 20A, 20B, and then the housing parts can be assembled. The fill material 36 in the ear bud helps to block and dampen acoustic vibrations which would otherwise be transferred to the ear canal.

It will be appreciated that the fill material 36 can be installed in other ways. For example, the housing of the ear bud can be constructed to have an opening into which the fill material is injected to fill voids inside the housing. The opening can be closed by silicone (or other substance) or by a cover installed in or over the opening after the fill material is injected.

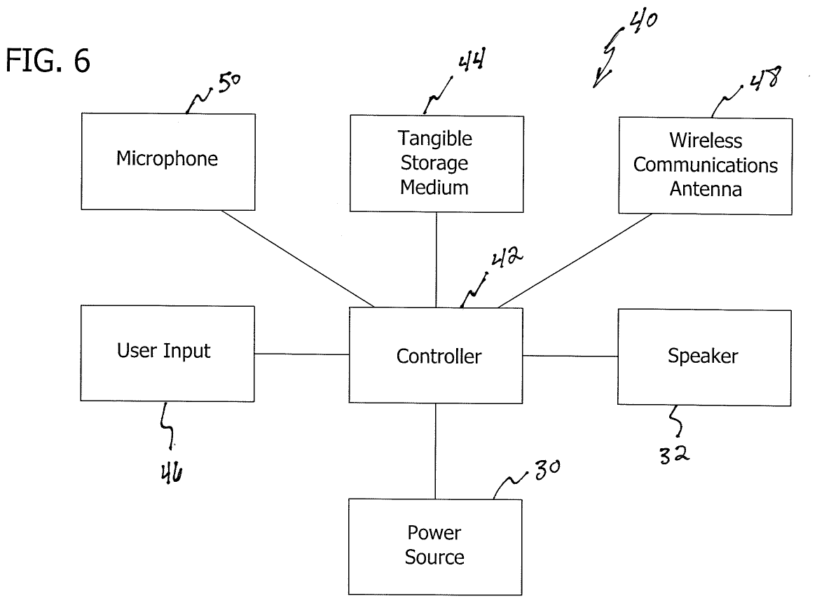

In one example, the electronic ear bud 10 can be used to block loud ambient sound from entering the ear canal of the user to protect the user, and to deliver desired sound (e.g., reproduced ambient or environmental sound, audio from a telephone call, or music from a smart telephone) to the ear drum. To enable such functionality, the ear bud 10 can include a control system 40 such as shown in FIG. 6. The control system 40 includes an earwear controller 42 (e.g., microprocessor or central processing unit), a non-transitory tangible storage medium 44 (e.g., including forms of storage such as software and/or firmware), and a user input 46. The control system 40 also includes a wireless communications antenna 48 (e.g., for Bluetooth communication) and a microphone 50. Moreover, the control system 40 includes the battery 30 and the speaker 32. The battery 30 provides electrical power to the control system components. The control system 40 includes interconnection electronics (e.g., including electrical, fiber optic lines, and/or wireless communication devices) that operatively connect the various components of the control system with each other and with other components of the earwear. For example, the earwear controller 42 can receive ambient sound signals from the speaker 32 and signals from the user input 46 via the interconnection electronics.

A printed circuit board assembly 60 in the housing 20 can be configured to include the controller 42 and the storage medium 44. The controller 42 is configured to read and execute instructions stored in the storage medium 44, and is responsive to the user input 46, for controlling operation of the earwear. A user can enter and/or modify instructions stored on the storage medium 44 via the user input 46. For example, the user input 46 could be a button or other actuator to turn the ear bud 10 on/off.

The arrangement is such that ambient or environmental sound waves are detected by the microphone 50 and monitored by the controller 42 according to instructions stored on the tangible storage medium 44. If the sound is higher than a threshold decibel, the sound is not transmitted to the user's ear canal via the speaker 32. However, if the sound is lower than a threshold decibel, then the sound is reproduced by the speaker 32 to be delivered to the user's ear canal. The sound delivered via the speaker 32 can be at a decibel level deviating from (e.g., greater than or less than) or the same as the original ambient sound. The arrangement is such that the user's ear is protected from loud noises such as gunfire, but the user is able to hear, via the microphone 50 and speaker 32, people talking on the firing range.

One or more components of the control system 40 could be omitted without departing from the scope of the present disclosure. For example, the microphone 50 may be omitted. Moreover, the control system 40 may lack the ability to selectively reproduce ambient sound, and the electronic ear bud 10 may be intended only for playing music or permitting a user to hear audio from their smart phone via the wireless communications antenna (e.g., Bluetooth connection).

It will be apparent that modifications and variations are possible without departing from the scope of the invention defined in the appended claims. For example, it will be understood that aspects of the disclosure can be implemented in other types of earwear or ear covers without departing from the scope of the present invention. For example, the earwear can be configured as a different type of ear plug.

As various changes could be made in the above constructions and methods without departing from the scope of the invention, it is intended that all matter contained in the above description and shown in the accompanying drawings shall be interpreted as illustrative and not in a limiting sense.

What is claimed is:

1. An electronic ear plug for protecting an ear drum of a human from ambient sound waves, the electronic ear plug comprising:
a housing defining an interior;
a tip supported by the housing, the tip configured to be received in an ear canal of the human;
a sound wave passage extending through the ear tip;
a speaker in the housing configured to emit sound waves to be delivered through the sound wave passage toward the ear drum;
a sealing material in the housing at least partially filling the housing, the sealing material configured to obstruct sound waves not emitted by the speaker from entering the sound wave passage from inside the housing.

2. An electronic ear plug as set forth in claim 1, wherein the sealing material is resiliently compressible.

3. An electronic ear plug as set forth in claim 1, wherein the sealing material overlies a side of the speaker opposite a portion of the sound wave passage into which the speaker is configured to emit sound waves.

4. An electronic ear plug as set forth in claim 1, wherein the sealing material comprises a stiffened caulk.

5. An electronic ear plug as set forth in claim 1, wherein the sealing material comprises silicone.

6. An electronic ear plug as set forth in claim 1, wherein the housing defines a head away from which the tip protrudes, the sealing material filling substantially all of the head.

7. An electronic ear plug as set forth in claim 1, wherein the sealing material forms a peripheral seal against the housing around the speaker.

8. An electronic ear plug as set forth in claim 7, wherein the sealing material covers a side of the speaker facing away from the sound wave passage.

9. An electronic ear plug as set forth in claim 1, wherein the sealing material fills gaps between the speaker and inside surfaces of the housing.

10. A hearing protection device for protecting an ear drum of a human, the device comprising:
a housing configured to be worn by the human;
a speaker in the housing, the speaker configured to emit sound waves toward the ear drum of the human when the housing is worn by the human;
a sound wave passage configured to permit passage of sound waves emitted by the speaker to the ear drum of the user when the housing is worn by the human; and
a sealing material in the housing at least partially filling the housing, the sealing material configured to obstruct sound waves not emitted by the speaker from entering the sound wave passage from inside the housing.

11. A hearing protection device as set forth in claim 10, wherein the sealing material is resiliently compressible.

12. A hearing protection device as set forth in claim 10, wherein the sealing material overlies a side of the speaker opposite a portion of the sound wave passage into which the speaker is configured to emit sound waves.

13. A hearing protection device as set forth in claim 10, wherein the sealing material comprises a stiffened caulk.

14. A hearing protection device as set forth in claim 10, wherein the sealing material comprises silicone.

15. A hearing protection device as set forth in claim 10, further comprising a tip connected to the housing, the tip being configured to be received in and to plug an ear canal of the human, the sound wave passage extending through the tip.

16. A hearing protection device as set forth in claim 10, wherein the sealing material forms a peripheral seal against the housing around the speaker.

17. A hearing protection device as set forth in claim 16, wherein the sealing material covers a side of the speaker facing away from the sound wave passage.

18. A hearing protection device as set forth in claim 10, wherein the sealing material fills gaps between the speaker and inside surfaces of the housing.

19. A method of manufacturing an ear plug, the method comprising:
arranging a speaker in a housing of the ear plug to emit sound waves through a sound wave passage of the ear plug;
installing sealing material in an interior of the housing, the sealing material forming a peripheral seal around the speaker and at least partially filling a void to a side of the speaker opposite the sound wave passage;
permitting the sealing material to stiffen.

20. A method as set forth in claim 19, further comprising closing the housing to capture the sealing material inside the housing.
